(19) United States
(12) Patent Application Publication
(10) Pub. No.: US 2008/0033423 A1
(43) Pub. Date: Feb. 7, 2008

(54) TOTAL VASCULAR OCCLUSION TREATMENT SYSTEM AND METHOD

(76) Inventor: James C. Peacock III, San Carlos, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/833,075

(22) Filed: Aug. 2, 2007

Related U.S. Application Data
(63) Continuation of application No. PCT/US2006/004222, filed on Feb. 2, 2006.
(60) Provisional application No. 60/649,506, filed on Feb. 2, 2005.

(51) Int. Cl. A61B 18/14

(52) U.S. Cl. 606/34; 606/41; 607/116

(57) ABSTRACT

A system is provided for providing vascular access across chronic total occlusions, in particular those that are particularly long such as in the lower periphery. A guide wire has an off-set, tilted tip section that provides rotational micro-dissection through tight CTO lesions. An outer catheter sheath prevents binding of the wire via wire reinforced composite polymeric construction. The outer sheath catheter includes an ablative outer surface for ablating tissue in contact therewith. The guide wire and outer sheath catheter are each driven by an actuator for cooperative advancement through the CTO. Rotational couplers rotate them, which may be at different speeds and via different couplers. The engagement of the wire within the sheath may allow for at least limited longitudinal movement between them during CTO advancement. Aspiration of ablated debris around the outer rotational ablation catheter is accomplished through suction ports through the composite wall and between adjacent windings of the reinforcement. Long CTOs of the peripheral vasculature are in particular benefited by the assembly, which allows on-going force transmission to the distal components through long portions of blockage, and allows for pilot lumen formation for advancing other interventional tools.

### CROSS-REFERENCE TO RELATED APPLICATIONS

This application claims priority from, and is a 35 U.S.C. § 111(a) continuation of, co-pending PCT international application serial number PCT/US2006/004222, filed on Feb. 2, 2006, incorporated herein by reference in its entirety, which claims priority from U.S. provisional application Ser. No. 60/649,506, filed on Feb. 2, 2005, incorporated herein by reference in its entirety.

This application is related to PCT international Publication No. WO/2006/084256, published on Aug. 10, 2006, incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37° C.F.R. § 1.14.

### BACKGROUND OF THE INVENTION

1. Field of the Invention

This invention relates to the field of medical devices, and more particularly to a catheter and guidewire system and method for crossing and treating total vascular occlusions via percutaneous translumenal procedures.

2. Description of Related Art

Total vascular occlusions, and more particularly chronic total occlusions (“CTO”), have long been considered one of the most significant challenges to percutaneous vascular interventional therapies. Recanalization of CTO's remains a leading indication for invasive open heart surgery, unapproachable by most less invasive catheter therapies.

It is believed that total occlusions generally occur with an initial, gradual occlusive progression of atherosclerosis. Eventually, as the vessel gradually narrows in the region, hemodynamics become critical. At some critical level of progression, clot forms as an initially complete occlusion, often upon a “rupture” of the underlying occlusion in response to the poor hemodynamic environment. Thus, what was once a progressively tight occlusion becomes a total occlusion. In either case, the total occlusion is typically characterized by at least three different types of tissues. Two of these types of tissues are: (1) smooth muscle tissue of the vessel wall; and (2) the atherosclerotic occlusion. “New” total occlusions, typically less than about 3 months old and often as much as 6 months old, are often characterized by a third type of tissue that is a readily definable clot in what was the “last true lumen.” In the “chronic” setting of CTO's, this once defined, relatively fresh clot region typically progresses to a more fibrotic form, often including a fibrous “cap” formed at the proximal/upstream extent of the old lesion.

CTO's of the coronary arteries represent a frequent reason that cardiology patients are either contra-indicated for, or otherwise fail, treatment by percutaneous translumenal approaches. Thus, CTO's are a frequent cause for patients to be referred instead to the highly invasive (and increased morbidity) option of open heart coronary artery bypass surgery. CTO's of the peripheral arteries in the lower extremities, e.g. legs, also represent one of the most frequent reasons patients undergo elective limb amputation. Whereas the coronary CTO experience is often characterized by occlusions that may be 1 to 3 centimeters long, the peripheral condition may be much more progressed and characterized by CTO lesions that may be 10 or even 20 centimeters in length. This difference results for a number of reasons. In one regard, peripheral vascular disease is often accommodated by the body by an ability to naturally “bypass” the occlusion via a substantial collateral blood flow network, wherein the blood flow is diverted to other branches at higher downstream flow rates that often share perfusion targets with the occluded vessel. In contrast, the heart, while also providing a collateral network, it is less developed. In another regard, the heart is more sensitive to compromised flow than the legs, and thus earlier progression of disease becomes critically symptomatic—the peripheral vessels thus may progress for much longer periods of time before symptoms become critical for locomotion, etc. In still another mode differentiating the types of lesions, peripheral vascular CTO's are often also characterized as being much more fibrotic and even calcified than the coronary counterparts, yet another mode of a more substantially progressed disease state. In any event, despite the cause-and-effect relationships, these differences in progression, length, and morphology of CTO's between coronary and peripheral vascular settings are well recognized.

The extreme measures of open heart surgery or leg amputation, respectively, for coronary and peripheral CTO's are unfortunate when so many percutaneous translumenal systems and treatment methods have emerged for treating vascular occlusions with significantly reduced invasion and morbidity, in fact sometimes even done on an “out patient” basis.

For example, angioplasty has now been a long accepted percutaneous translumenal intervention for treating vascular occlusions, wherein a balloon is placed within a vessel along a location that is occluded and then expanded to mechanically apply a controlled injury to reopen the occlusion.

Another example of a well developed, percutaneous translumenal vascular occlusion treatment includes atherectomy or ablation of vascular occlusions. This alternative generally involves destroying the matrix of the occlusion sufficiently for it to be removed from the area of occlusion and thus reopen the vessel with none or significantly reduced remains of the occlusive lesion.

Some such previously disclosed atherectomy or ablation systems and techniques include devices having distal surfaces that are forced against a lesion from an upstream position in the native vessel in order to initiate the atherectomy/ablation procedure.

One such device, for example, provides an assembly of cutting blades that extend around a radius along a distal face of a rotating housing. The rotating blades are forced distally against the lesion to begin the cutting process, and suction is provided to remove the ablated debris proximally into the device through openings between the blades.

At least one other previously disclosed device and method uses an abrasive distal surface on a high speed spinning burr that is forced against the lesion. In one particular commercial product, the burr is metal and has a tapered distal surface that is coated with sharp diamond particles. This surface is believed to be selectively ablative to harder tissues, such as calcifications or fibrous tissue, when spun and forced distally against a vascular occlusion. Such technique has been observed to produce ablated debris of such small diameter that often it is merely allowed to flow downstream of the lesion into the downstream vascular bed where it is either cleared, assimilated, or otherwise may form downstream vessel occlusion(s) but generally to only small vessels such as capillaries or veinules. Other more recent developments have been disclosed that include applying suction through apertures in the distal wall of the abrasive burr in order to remove the ablated debris from the vascular blood flow.

At least one other atherectomy device and method has been disclosed that requires positioning the atherectomy device within a lumen through the lesion in order to cut and remove the blockage. This device includes a housing with an open window into a channel through which a cutting blade may be advanced. An expandable balloon is positioned opposite the open window. By expanding the balloon on one side of the device within the lesion, material from the lesion is forced within the channel where it is cut by the blade and suctioned out proximally through the device.

The advent of these percutaneous translumenal solutions to coronary artery disease is widely acclaimed as marking one of the most revolutionary and beneficial changes in modern medicine. However, such methods have been associated with as high as about 30% “restenosis” rates, wherein the body's own response to the controlled balloon or ablation injury scars and reblocks the vessel, sometimes to a worse condition than before the intervention. Thus, a more recent and significant development has included the introduction of intravascular stents.

Intravascular stents are generally expandable tubular cages constructed of a web of interconnecting struts, and are typically either self expanding (e.g. nickel-titanium shape memory alloy) or expandable by a balloon located within the stent's tubular wall. In either case, the stent is delivered in a collapsed condition to a lumen within the lesion and is then expanded and implanted against the interior surface of the lesion to hold it open. Stenting may be done either during recanalization, such as during angioplasty by placing the stent with the angioplasty balloon, or after recanalization such as after an atherectomy or other ablation procedure.

Stenting has become the convention for percutaneous translumenal treatment of vascular occlusions, in particular coronary interventions, and has generally been observed to reduce restenosis rates to generally about 20% or less. Notwithstanding this improvement over non-stented interventions (e.g. 20% vs. 30% restenosis), still further advancements have been investigated in recent years that provides bioactive agents coated onto stents that act as “anti-restenosis” agents. Some preliminary clinical data has suggested that certain combination(s) of stent and anti-restenosis compound may reduce restenosis rates to as low as 10% or less.

All of these treatments however share a common obstacle that has prevented their significant use in treating total occlusions—they all generally require some remaining lumen through the occlusion in order to perform the recanalization function (or “open the vessel”) as intended.

More specifically, in one regard, known percutaneous translumenal vascular occlusion treatments typically require use of a “guidewire.” A typical guidewire used in these interventions is generally constructed as a long, thin metal wire with a distal end portion having shaped, torqueable, radiopaque tip. The guidewire's distal end portion is initially steered through the vascular tree to the occlusive lesion, via manipulation of the guidewire's proximal end extending outside of the patient and also using x-ray or fluoroscopic visualization of the radiopaque tip in the vessels viewed against a radiopaque dye-enhanced roadmap of the vascular tree. The guidewire is then placed through and across the occluded region of the vessel to be treated. Once so positioned, the treatment device(s) are adapted to ride over the guidewire, via a guidewire lumen, and then follow the seated guidewire, using it as a “rail”, as means to position at and through the lesion in order to perform the desired dilatation or recanalization treatment there.

Angioplasty balloons, stents, and some atherectomy devices as noted above, further suffer from the requirement that they be positioned in a lumen within the lesion in order to perform their job to open or recanalize the area. Balloons and stents must be so positioned in order to thus be radially expanded to dilate or hold the area open, respectively; whereas atherectomy devices typically require the occlusion to be seated within the cutting housing via radial force from the opposite balloon. Others of the previously disclosed ablative devices that function by distal advancement against the lesion from a proximal location do not suffer from this requirement to pass into the lesion first. However, even these devices and related techniques still typically require the guidewire as a rail to direct the ablation process through the lesion else it may go astray and cause unintended and dangerous damage through the vessel wall.

In general, various different types of guidewires have been previously developed and commercialized to meet the different needs of particular conditions amongst the patient population with occlusive disease. Typical guidewires used in coronary inventions have diameters that are generally 0.010″, 0.014″ (most prevalent), 0.016″, or 0.018″; guidewires used in treating peripheral artery occlusive disease such as in the legs are often as big as 0.035″ in diameter. In another regard, various different wires of varied respective stiffness (or, conversely, “floppiness”) are commercially available.

The most typical type of guidewire of choice for crossing, and thus allowing for treatment of, total occlusions are those of relatively stiffer construction, (e.g. “standard” guidewires). The general goal of crossing a guidewire through a total occlusion is to find the last true lumen; however, other paths are frequently found, such as along the vessel wall, or merely breaking through and across the atherosclerotic tissue of the occlusion. The choice of a stiffer wire allows for a “brute force” approach to pushing or dottering across the lesion. Some physicians prefer use of smaller (e.g. 0.010″ for coronary interventions) and more floppy wires as the first choice for CTO's, based upon the ability to better follow a small remnant lumen. Often, multiple types of wires are tried in series, as different lesion morphologies or anatomical tortuosities respond different to different types of wires. In any case, where some slight remnant of a last true lumen may be found by such a guidewire tip, conventional guidewire crossing techniques are often successful, in particular in the hands of a highly skilled and experienced physician. This is often the case for more recent or “new” total occlusions. In other cases, however, all attempts fails. And, in certain circumstances, more injury may have been caused by the failed attempt, such as either causing a dissection in the vessel wall that may propagate upstream to a more proximal (and thus more dangerous) area of instability, or by perforating the vessel wall with the wire which may cause blood loss that may lead to tamponade.

According to the shortcomings of conventional devices in allowing for total occlusions to be treated, various devices and methods have been previously investigated that are intended to enhance the ability to recanalize CTO's and thus reperfuse downstream ischemic tissues in patients using percutaneous translumenal techniques.

At least one previously disclosed system and method is intended to puncture through a totally occluded artery proximal of the total occlusion, and provide a shunt through the puncture and into another puncture site into an adjacent vein. This technique is done in order to direct the arterial flow through the shunt to replace the venous flow with the higher pressure arterial flow, and thus use the vein for a flow conduit into the downstream ischemic tissue. In some regards, this may be simply a retrograde flow path into the tissue via the vein that naturally conducts flow in the opposite direction than it its flow during this artificial shunting. In other techniques, a further series of punctures are made back from the vein and back into the artery downstream of the CTO, thus simply shunting around the CTO via a segment of the vein. In either case, however, the intentional arterial and vein perforation technique is an aggressive approach having inherent risks of internal blood loss, in addition to the possibility that the perforation could lead to further unwanted wall injury such as a propagating dissection or “scarring” consistent with a restenosis condition. In addition, this technique assumes (1) that the vein may be found using the percutaneous translumenal approach under X-ray guidance; and (2) in other regards, that the retrograde flow through the vein once found and successfully shunted will provide retroperfusion to the same tissue that was critically lacking blood due to the total occlusion. Moreover, the techniques share the assumption that a vein is located conveniently adjacent the CTO to be bypassed.

Several other devices and methods have been investigated that are intended to enhance the basic guidewire crossing procedure through the occluded area. At least one such device uses ultrasonic energy applied to a guidewire in order to enhance its ability to propagate along a desired path through the occluded area, hopefully through the last true lumen. At least one other disclosed device and method applies a machine-aided mechanical force to the wire intended to improve on the manual forces of conventional guidewire crossing techniques. Such machine-aided forces have included rotation as well as reciprocating longitudinal forces. At least one other device and method intended to provide a wire with enhanced crossing ability for total occlusions includes an enlarged tip in order to provide enhanced dottering forces through the lesion. Alternatively, use of wires with reduced tip diameter has also been investigated for crossing particularly tight lesions. At least one such guidewire has been previously disclosed having a proximal diameter of 0.014″ and a distal diameter along the tip of 0.010″.

In the case of relatively “new” total occlusions, these previously disclosed machine-enhanced techniques of spinning, sonicating, or reciprocating a guidewire may provide for some improvement over and share some general success with conventional manual techniques. However, in the case of CTO's, the previously disclosed techniques have been largely unsuccessful in achieving a predictable ability to cross into the true lumen downstream of the lesion. Many patients are thus generally left untreated, partially ameliorated with drugs, or are referred to either bypass surgery or limb amputation.

In addition to the foregoing, even if a rail is successfully positioned across a CTO and into the native downstream vessel, the significant amount of blockage material associated with the CTO presents a particular additional challenge to achieving substantial and successful recanalization. In one regard, a CTO will still remain extremely tight over even a successfully crossed guidewire, and further crossing with an angioplasty balloon, stent device, or atherectomy device may still present a formidable challenge. Moreover, angioplasty and stenting techniques require pushing the occlusive material aside, ideally to the extent that the once occluded area is dilated to a diameter matching the adjacent upstream and downstream vessel wall. However, such extensive dilatation of a CTO is difficult to achieve, and CTO's are often “under” dilated. In addition, the significant volume of repositioned material may cause further complications post-operatively. Even ablation devices that benefit from an advancement mode of operation against the lesion from an upstream position in the native vessel may still be substantially challenged by the morphology of a tightly fit lesion down over the guidewire.

Adjunctive applications of atherectomy followed by balloon angioplasty and/or stenting have also been disclosed and may be useful for treating a CTO once a guidewire is crossed. In one regard, a “pilot” channel may be made with a “distal advancement” type of atherectomy/ablation device. Such device may then be removed and followed by angioplasty/stenting. However, these devices are typically extremely expensive disposable articles, and some such devices require particular guidewires for operation that may not otherwise be the physician's guidewire of choice. Moreover, they are generally not designed merely for this pilot channel use, and thus may be more ablative than necessary or even desired to merely achieve sufficient clearance to pass a balloon or stent (particularly if undesirable downstream debris results).

In the entire field of CTO devices and methods, it is also generally the case that solutions have been investigated for the principal target of CTO's of the coronary vessels. Prior disclosures often apply similar criteria and designs to the intended use in both the coronary and peripheral CTO conditions as similar challenges. However, the differences in anatomy and lesion morphology between these conditions are substantial. In particular, solutions for the longer, more progressed, and typically straighter peripheral vascular CTO's may not be appropriately leveraged from devices and methods principally intended for coronary CTO's. For example, the typically straighter and larger diameter anatomy of the peripheral vessels may allow for solutions that might not be safe or efficacious for the shorter, more tortuous vessels of the coronaries. In one particular regard, the extensive length of many peripheral CTO's will present substantial binding on guidewire devices even if such a guidewire is able to initiate a progression through the proximal entrance into the CTO. More specifically, the ability to transfer forces to a guidewire tip even 1 to 2 centimeters buried into a CTO may be sufficient for crossing a coronary CTO; such achievement may be only 10 percent along the way to getting through a peripheral CTO, and it is the frequent condition that the ability to apply force to the wire tip diminishes substantially with further advancement through a tight lesion.

There is still a need for an improved percutaneous translumenal system and method for placing a guide rail across a chronic total vascular occlusion and into the native artery lumen downstream of the occlusion.

There is also still a need for an improved percutaneous translumenal system and method for providing a pilot channel through a total vascular occlusion in order to allow recanalization, dilatation, or stent devices to be positioned within the lesion for subsequent treatment.

There is also still a need for improved percutaneous translumenal system and method adapted to specifically treat peripheral vascular CTO's in particular.

### SUMMARY OF THE INVENTION

The invention according to one aspect is a CTO crossing system that includes an assembly with an inner wire and a cooperating outer sheath catheter. The inner wire is adapted to couple to an actuator that spins the inner wire. The inner wire has a distal tip that is offset from the longitudinal axis of rotation for the wire's core. Accordingly, the distal tip is adapted to auger through a CTO lesion upon spinning and advancement of the sheath/wire assembly. The sheath is adapted to generally be advanced through the CTO lesion immediately behind the wire's augering distal tip, and is constructed to resist binding of the spinning wire by substantially tight CTO tissue as the wire assembly is advanced through the lesion.

In one further mode of this aspect, the tip of the wire has a radial enlargement with a length along a longitudinal axis that is canted such that the longitudinal axis of the wire tip is not parallel to the longitudinal axis of rotation of the proximal core wire. In one beneficial embodiment, the distal end of the radial enlargement is located along the longitudinal axis of rotation, but the proximal end of the enlargement is offset from the rotational axis, such that the proximal end rotates about a radius around the axis of rotation. This beneficially provides the desired augering affect to separate tissue in the path of the advancing wire assembly while maintaining the distal most tip substantially centrally along the axis of advancement, thereby assisting the wire assembly to be maintained within the lesion during advancement.

Another aspect of the present disclosure includes an ablative sheath that is adapted to be advanced into and along a CTO lesion over a guide rail and to rotationally ablate the CTO lesion tissue radially surrounding an outer ablative surface of the sheath. One beneficial further mode of this aspect includes suction ports and the sheath is adapted to couple to a vacuum source in order to suction withdraw ablated debris from the radial area surrounding the rotational ablative outer surface of the sheath.

Another aspect of the invention is a CTO treatment system that includes a spinning CTO guide wire in combination with a rotational atherectomy device that includes an abrasive surface which is spun against the CTO lesion material to ablate it into loose debris. In one beneficial further mode of this aspect, suction ports are positioned relative to the abrasive surface and coupled to a vacuum source such that the ablated debris may be removed.

Another aspect of the invention is a CTO crossing system with a crossing guidewire that is adapted to proximally couple to a rotational housing of a motorized rotation actuator in such a manner that rotation of the crossing guidewire by the actuator is preventing from exceeding a predetermined resistance force by releasing the wire from an applied rotational force at the predetermined level.

In one beneficial mode of this aspect, the coupling between the wire and motorized rotation actuator is constructed to provide for an interference between a rotational housing of the actuator and a proximal coupler on the guidewire. The interference is designed to fail at a particular force level to allow the wire to slip within the rotational housing. In one further beneficial embodiment, this controlled interference failure is achieved with at least one polymeric rib located on either the wire coupler or the rotational housing and that provides at least in part for the mechanical interference for rotational coupling but exhibits an elastic yield at the predetermined force, thus resulting in the slipping.

In another beneficial mode of this aspect, the controlled rotational housing is coupled to a mechanical clutch mechanism associated with the motor of the actuator. The clutch mechanism may be mechanically constructed to slip at the predetermined force level. Or, a sensor may be included in the actuator assembly and a control unit coupled to the motor may be programmed to shut off the motor, or actuate a clutch, at a predetermined measured force level.

Another aspect of the invention is a rotational ablation atherectomy device that includes an ablation assembly having an adjustable effective ablation diameter. In one particular mode, the ablation assembly includes a housing with a distal surface that includes an abrasive surface adapted to ablate CTO tissue upon rotational engagement with such tissue. In one particular embodiment, the housing includes a polymeric surface with abrasive particles secured thereto. In one variation, the abrasive particles may be partially embedded within the polymeric surface, such that an abrasive portion of the particles are exposed over the surface. In still further features, the particles may be diamond. In another feature, the polymer may be elastomeric, and may be in particular beneficial features a silicone, polyurethane, or latex material.

In another particular beneficial variation, the housing includes a polymer composite with a support structure, which may be in further beneficial features a wire reinforcement such as a braid or coil imbedded within the polymer.

Another aspect is a medical device system for providing vascular access across a chronic total occlusion (CTO). This includes a catheter actuator and a catheter configured to be actuated by the catheter actuator, and with a first elongate body having a proximal end portion, a distal end portion, and a guide wire lumen extending between a proximal port and a distal port located at the distal end portion. A wire actuator is also provided with a guide wire configured to be actuated by the wire actuator. The guide wire has a second elongate body with a proximal end portion, a distal end portion with a first longitudinal axis and first outer diameter, and a distal tip section on the distal end portion with a second outer diameter that is radially enlarged relative to the first outer diameter. The catheter is adapted to moveably engage the guide wire in a crossing configuration with the guide wire extending within the guide wire lumen and through the proximal and distal ports with the enlarged distal tip section located externally of the guide wire lumen distally beyond the distal port. The actuated catheter and the actuated guide wire are configured to advance across the CTO substantially together in the crossing configuration.

Another aspect is a medical device system for providing vascular access across a chronic total occlusion (CTO) in a body of a patient. This includes a catheter with a first elongate body having a proximal end portion, a distal end portion comprising a wire reinforced polymeric wall, and a guide wire lumen extending between a proximal port and a distal port located at the distal end portion. A wire actuator is provided with a guide wire with a second elongate body with a proximal end portion, a distal end portion with a first longitudinal axis, a distal tip section on the distal end portion, and that is adapted to be actuated by the wire actuator. The catheter is adapted to moveably engage the guide wire in a crossing configuration with the guide wire extending within the guide wire lumen and through the proximal and distal ports with the distal tip section located externally of the guide wire lumen distally beyond the distal port. The catheter and the actuated guide wire are configured to advance across the CTO substantially together in the crossing configuration and with the wire reinforced distal end portion of the catheter configured to resist radial binding of the CTO onto the distal end portion of the actuated guide wire.

Another aspect is a medical device system for conducting a medical procedure related to a medical condition within a body of a patient. This aspect includes a first mechanically actuated device comprising a catheter with a first elongate body having a first proximal end portion and a first distal end portion. A second mechanically actuated device is also provided and includes a second elongate body with a second proximal end portion and a second distal end portion. A lumen extends within the first actuated device between a proximal port and a distal port at the distal end portion. The second actuated device is located at least in part within the lumen with the second distal end portion extending from the lumen through the distal port in a delivery configuration. In the delivery configuration, the first and second distal end portions are adapted to be delivered across a resistance to a location within the patient's body with the first and second proximal end portions, respectively, extending externally from the patient.

Another aspect is a medical device system for providing vascular access across a chronic total occlusion (“CTO”) within a body of a patient. This aspect includes a catheter comprising a first elongate body with a proximal end portion, a distal end portion, and a guide wire lumen with a distal port at the distal end portion, and a guide wire having a second elongate body with a proximal end portion and a distal end portion that extends along a first longitudinal axis with a first outer diameter. A distal tip section is located on the distal end portion of the guide wire. The distal tip section has a second outer diameter and a length along a second longitudinal axis between a proximal end and a distal end. The second longitudinal axis is angled relative to the first longitudinal axis. The second outer diameter is greater than the first outer diameter such that the distal tip section is radially enlarged relative to the distal end portion of the second elongate body. The second elongate body of the guide wire is configured to be rotatably disposed at least in part within the guide wire lumen in a crossing configuration with the distal tip section of the guide wire extended externally of the guide wire lumen distally from the distal port. The guide wire is torquable such that upon rotation of the proximal end portion externally of the patient's body such that sufficient torque is transmitted to the distal tip section at a CTO location within the patient's body so as to rotate the distal tip section about the longitudinal axis of the guide wire's distal end portion. In addition, the distal end portion of the first elongate tubular body and the guide wire are adapted to cooperate in coordinated advancement across the CTO in the crossing configuration. The distal end portion of the first elongate tubular body is constructed so as to substantially inhibit resistance from the CTO on the torque transmission from the guidewire proximal end portion to the distal tip section during the coordinated advancement of the guide wire and the distal end portion of the first elongate tubular member through the CTO in the crossing configuration.

Another aspect is a medical device system for providing vascular access across a chronic total occlusion (“CTO”) in a body of a patient. This includes a catheter having a first elongate tubular body with a proximal end portion, a distal end portion with a length along a longitudinal axis, a guide wire lumen adapted to moveably engage a guide wire at least in part along the distal end portion, and a suction lumen extending between a proximal port along the proximal end portion and a distal port at the distal end portion. The distal end portion comprises a wire reinforced polymeric composite tubular member with spaced portions of wire coupled with a polymeric wall and also with an outer surface located along a circumference around the longitudinal axis. The proximal end portion comprises an ablation coupler that is adapted to couple to an ablation actuator. The composite tubular member is coupled to the ablation coupler and is adapted to ablate CTO tissue in contact with the outer surface upon actuation by an ablation actuator coupled to the ablation coupler. The distal port is located proximally of the distal tip and through the polymeric wall between the spaced portions of wire of the wire reinforced polymeric composite tubular member. The proximal port comprises a vacuum coupler that is adapted to couple to a vacuum source. Accordingly, by coupling the vacuum coupler to an actuated vacuum source, sufficient suction is applied at the distal port to remove debris of CTO tissue ablated by the tubular member.

Another aspect is a medical device system for providing vascular access across a chronic total occlusion (“CTO”) in a body of a patient. This includes a catheter having a first elongate tubular body with a proximal end portion, a distal end portion comprising a tubular member with an inner surface that defines a lumen along a longitudinal axis and an abrasive outer surface located along a circumference surrounding the longitudinal axis, and also with a guidewire passageway defined extending at least in part through the lumen of the tubular member. The proximal end portion comprises an ablation coupler that is adapted to be coupled to a rotational ablation actuator. The first elongate tubular body is sufficiently torqueable such that the tubular member is rotable within a CTO within the patient's body by rotating the proximal end portion with a rotational ablation actuator located externally of the patient's body. Accordingly, by rotating the tubular member within the CTO the abrasive outer surface is adapted to mechanically ablate CTO tissue in contact therewith sufficient to aid the catheter in advancement through the CTO.

Another aspect is a medical device system for removing soft tissue from a body space within a patient. Included is a catheter having a first elongate tubular body with a proximal end portion, a distal end portion with a length along a longitudinal axis and terminating in a distal tip, and a passageway extending between a proximal port along the proximal end portion and a distal port along the distal end portion. The distal port is located proximally of the distal tip and through the elongate tubular body. The proximal port comprises a proximal coupler that is adapted to couple to a source of vacuum pressure. The proximal port is fluidly coupled to the distal port such that upon coupling the proximal port to an actuated source of vacuum pressure suction is applied at the distal port. The proximal end portion further comprises an ablation coupler adapted to couple to an energy source. The distal end portion further comprises an ablation assembly coupled to the ablation coupler. The ablation assembly is adapted to be actuated by an energy source coupled to the ablation coupler so as to emit sufficient energy into soft tissue located within the passageway to ablate the tissue without substantially ablating other tissue located externally of the passageway.

Another aspect is a medical device system that includes, in one regard, a first elongated body with a proximal end portion, a distal end portion that is adapted to be positioned within a patient's body with the proximal end portion extending externally from the patient, and a wall with an elastomeric material and an outer surface along the distal end portion. A plurality of abrasive particles is provided along the outer surface. Each of the abrasive particles comprises a first portion that is embedded within the elastomeric material below the outer surface and a second portion that extends above the elastomeric material from the outer surface. Accordingly, by actuating the distal end portion into motion within the patient's body the abrasive particles are configured to mechanically ablate tissue in contact with the outer surface.

Other highly beneficial aspects and modes and embodiments are further contemplated though not specifically provided in this section, including for example as further provided in the text below or claims provided herewith.

In one particular regard, it is to be further appreciated that the various methods herein shown and described constitute further aspects of particular benefit and invention. For example, a method of crossing a CTO lesion via a rotationally actuated guide wire inside of an outer protective catheter is one such exemplary method. Another example is a method for rotational microdissection via the actuated guidewire in combination with performing rotational atherectomy via the outer sheath catheter. Other methods are contemplated as apparent to one of ordinary skill based upon review of the totality of the present disclosure.

The systems and methods herein summarized may be provided together, or in separate component parts or steps and still provide significant value as further contemplated aspects of this disclosure. In addition, the various systems and related components described may be chosen from a kit of various sizes and specific embodiments in order to suit a particular medical need or patient anatomy, as would be apparent to one of ordinary skill based upon review of the totality of this disclosure. In addition, medical systems are often provided in sterile, packaged form, with packaging inserts describing the instructed methods for their use. These aspects are also considered further aspects of additional value, both independently and in combination with the various other aspects and modes described.

The invention further contemplates additional combinations and sub-combinations of the various embodiments, features, and variations herein shown and described as would be apparent to one of ordinary skill in the art based at least in part upon this disclosure.

### BRIEF DESCRIPTION OF THE SEVERAL VIEWS OF THE DRAWINGS

FIG. 1 shows a chronic total occlusion (CTO) crossing system according to the present invention.

FIG. 2 shows an alternative embodiment of the CTO crossing system according to the present invention, in which the core wire and outer sheath are tapers at a plurality of locations.

FIG. 3 shows an alternative embodiment of the CTO crossing system according to the present invention, in which the wire and sheath may be advanced independently of one another.

FIG. 4A is a side view of one configuration of a tip for a CTO crossing system.

FIG. 4B is an end view of the tip in FIG. 4A.

FIG. 5A is a side view of a second configuration of a tip for a CTO crossing system.

FIG. 5B is an end view of the tip in FIG. 5A.

FIG. 6A is a side view of a third configuration of a tip for a CTO crossing system.

FIG. 6B is an end view of the tip in FIG. 6A.

FIG. 7 is a side view of an alternative configuration of a tip for a CTO crossing system.

FIG. 8 shows one embodiment of a core wire for a CTO crossing system.

FIGS. 9A-9D are cross-sectional views of the core wire embodiment shown in FIG. 8.

FIG. 10 shows another embodiment of a core wire for a CTO crossing system.

FIGS. 11A-11B are cross-sectional views of the core wire embodiment shown in FIG. 10.

FIG. 12 shows an embodiment for adapting the wire aspect of the sheath and wire assembly for spinning rotation.

FIG. 13A shows the embodiment of FIG. 12 in conjunction with a collet assembly.

FIGS. 13B-13C is a cross-sectional view of the collet in FIG. 13A, showing open and closed positions, respectively.

FIGS. 14A-14C show various embodiments of the interface between outer housing and the collet adapter.

FIG. 14D shows an alternative embodiment of a keyed assembly, in which a square keyhole interface is employed.

FIGS. 15A-15C show various embodiments of proximal couplings, in which interfacing ribs are shown under yield during mechanical slippage at a particular force.

FIGS. 16A-16C are cross-sectional views of various embodiments of the outer tubular sheath in the CTO crossing system according to the present invention.

FIG. 17 is a cross-sectional view of an outer tubular sheath as in FIGS. 16A-16C, in which the outer layer has an abrasive coating.

FIG. 18A is a view of the coaxial space between the outer sheath and the internal sire, in which the space includes elongated ports.

FIG. 18B is a view of the coaxial space between the outer sheath and the internal sire, in which the space includes discrete ports.

FIG. 19 shows an exemplary system according to the present invention, in which an actuator assembly is present for rotating the wire and outer sheath.

### DETAILED DESCRIPTION OF THE INVENTION

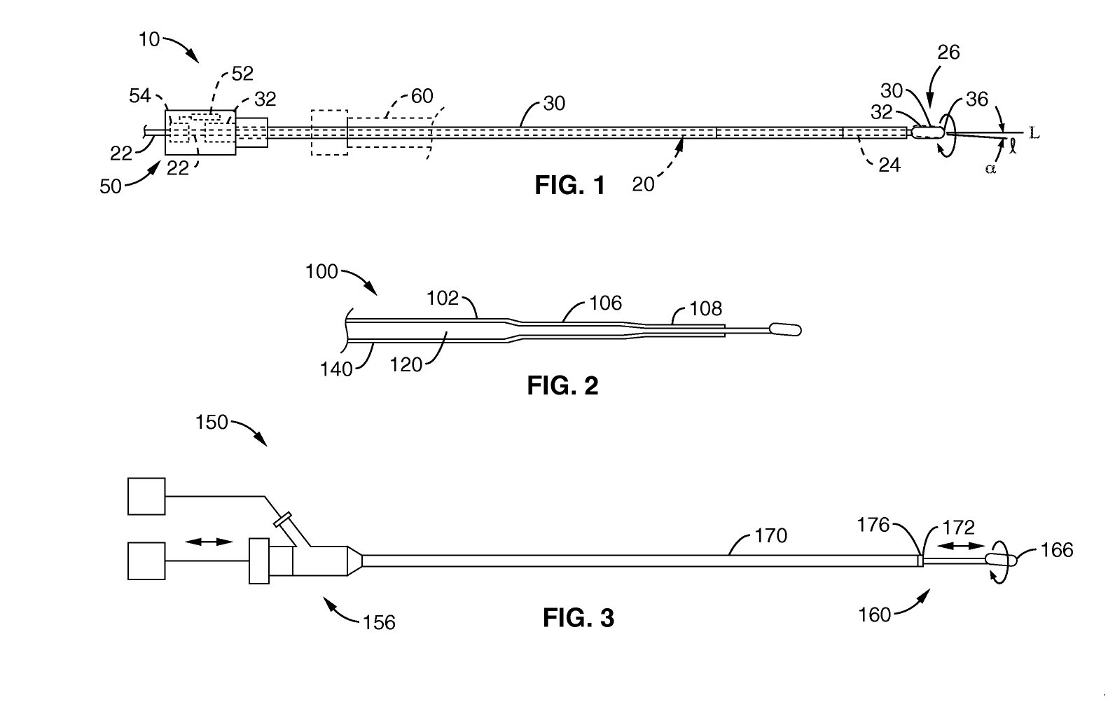

As shown in FIG. 1, the invention includes a chronic total occlusion (CTO) crossing system 10 with a wire 20 located coaxially within an outer tubular sheath 40. The wire includes a distal tip 26 extending beyond the distal end 36 of the tubular sheath. The proximal end portions 22, 32 of each of the wire 20 and tubular sheath 30, respectively, are coupled to an actuator assembly 50 in such a manner that the wire 20 is mechanically spun by a motor 52 coupled to the wire via a coupler 54 and so that the wire 20 spins within the outer tubular sheath 40.

The wire's distal tip 26 includes an enlargement 30 that, in the illustrative embodiment shown in FIG. 1, is constructed and oriented in a specific and particularly beneficial manner as follows. The enlargement 30 has a length along a longitudinal axis I that extends between a proximal end 32 and a distal end 36. The enlargement 30 is canted relative to a core 24 of wire 20 to which the enlargement 30 is secured such that longitudinal axis I is at an angle α relative to longitudinal axis L of the core wire 20. In addition, the distal end 36 is generally centered along longitudinal axis L and its proximal end 32 is offset relative to longitudinal axis L. Accordingly, by spinning the wire 20 around longitudinal axis L, enlargement 30 spins around a conical pattern centered around distal end 36 as the point where longitudinal axes I and L cross, and tapering proximally outward to a radius at proximal end 32. This motion, coupled with distal advancement through a CTO lesion, creates an oscillation designed to push tissue radially apart. This allows for the enlargement 30, starting with distal tip 36, to preferentially find and propagate along paths of least resistance to such motion, which is believed to most often occur at natural tissue planes between at least two amorphous tissues in the CTO, such as for example plaque and fibrous thrombus, or plaque and native vessel wall tissue.

The sheath 40 is designed to be tightly toleranced over the internally housed wire 20 such that the sheath 40 and wire 20 advance together through a CTO. This allows for the outer sheath 40 to shoulder the radial compressive forces of the CTO that would otherwise bind the core wire 20 once distal enlargement 30 is advanced substantially into a CTO. It is believed that without such outer sheath 40 the intended torsional rotation at the tip 26 may be compromised by substantially long CTO lesions, such as in the legs for example which may be as long as or even exceed about 10 centimeters, or even as much as or more than about 15 or 20 centimeters. Such binding may further cause torsional tension build up on the core 24 of wire 20 proximally of the distal enlargement 30, which under certain combinations of forces and without radial confinement within such an outer sheath 40, may result in a failure mode wherein the core 24 prolapses upon itself. This event may cause for example a significant remodeling of the wire 20 in the vessel, such as for example potentially causing a loop to form transverse to longitudinal axis L, which loop of substantially stiff material may cause damage to the proximal vessel.

After the assembly of the wire 20 and outer sheath 40 are advanced successfully across the CTO, the outer sheath 40 may be proximally removed from the wire 20, which now is able to act as a rail for a treatment device such as angioplasty, stent, or atherectomy or ablation (not shown). Alternatively, the sheath 40 may remain and itself provide for the coaxial rail over which treatment device(s) are tracked to and across the lesion.

The sheathed wire system 10 shown in FIG. 1 does not have a steering mechanism for advancing the assembly to the lesion through the vascular tree. This provides a benefit in that the distal tip 26 is optimized merely for lesion crossing, whereas the shaped distal tips intended to enhance steering of conventional steerable guidewires point “off-axis” and may preferentially advance off axis toward the vessel wall when forced longitudinally distally against a lesion. Nevertheless, the present assembly is generally advanced to the lesion of interest under fluoroscopic guidance and will often be provided with steering capabilities within an overall delivery system. Therefore, in one further embodiment a separate delivery sheath 60 (shown in shadow in FIG. 1) may be first advanced to the lesion over a first guidewire (not shown), which first guidewire is then removed and replaced with the sheathed wire assembly 10 of the present embodiment which tracks through the proximally positioned delivery sheath 60 and against the target CTO lesion.

FIG. 2 shows an alternative design 100 to that shown in FIG. 1, wherein both the core wire 120 and outer sheath 140 are tapered at a plurality of locations, which allows for stepwise or gradual reduction in diameter and stiffness. Proximal region 102 is larger and stiffer than intermediate region 106, which is larger and stiffer than distal region 108. This tapering design is adapted to enhance advancement of the assembly to and through a tortuous anatomy and lesion, respectively. However, in the event the coaxial engagement of these components is tightly toleranced, this generally makes removal of outer sheath 140 from wire 120 difficult once the wire/sheath assembly 100 is advanced across the lesion. Therefore, according to such particularly tightly toleranced embodiment, a subsequently delivered adjunctive treatment device will often be advanced coaxially over the sheathed wire assembly 100. In other embodiments though, the tapered construction(s) of the respective components may provide sufficient clearance to enable removal of the outer sheath 140 prior to using the exposed wire 120 as the delivery rail for subsequent recanalization tools.

FIG. 3 shows another embodiment 150 wherein the wire 160 and sheath 170 may be advanced independently of each other. A flush lumen 172 is provided to the coaxial space between the wire 160 and outer tubular sheath 170, and a proximal hemostatic valve 180 (which may be a removable separate accessory) on the sheath 170 allows the wire 160 to be independently advanced/spun/retracted within outer sheath 170 without substantial binding or loss of blood. This allows stepwise independent advancement of the wire 160 and outer sheath 170 through a tight CTO lesion, which may be helpful as the profile of the wire 160 is significantly reduced when extended distally from the tip 176 of outer sheath 170. In order to manage proper positioning, the distal tip areas 166, 176 of both the inner wire 160 and outer sheath 170, respectively, are provided with radiopaque markers. FIG. 3 also schematically illustrates a proximal coupler housing 156 with various proximal adapting features for actuating movement of the wire 160 relative to the outer sheath 170 (double headed arrows), as well as schematic representations for wire drive component and fluid communication via a side-arm adapter of housing 156, such as for suction of infusion of liquid materials, as would be apparent to one of ordinary skill upon review of the Figures and this accompanying description.

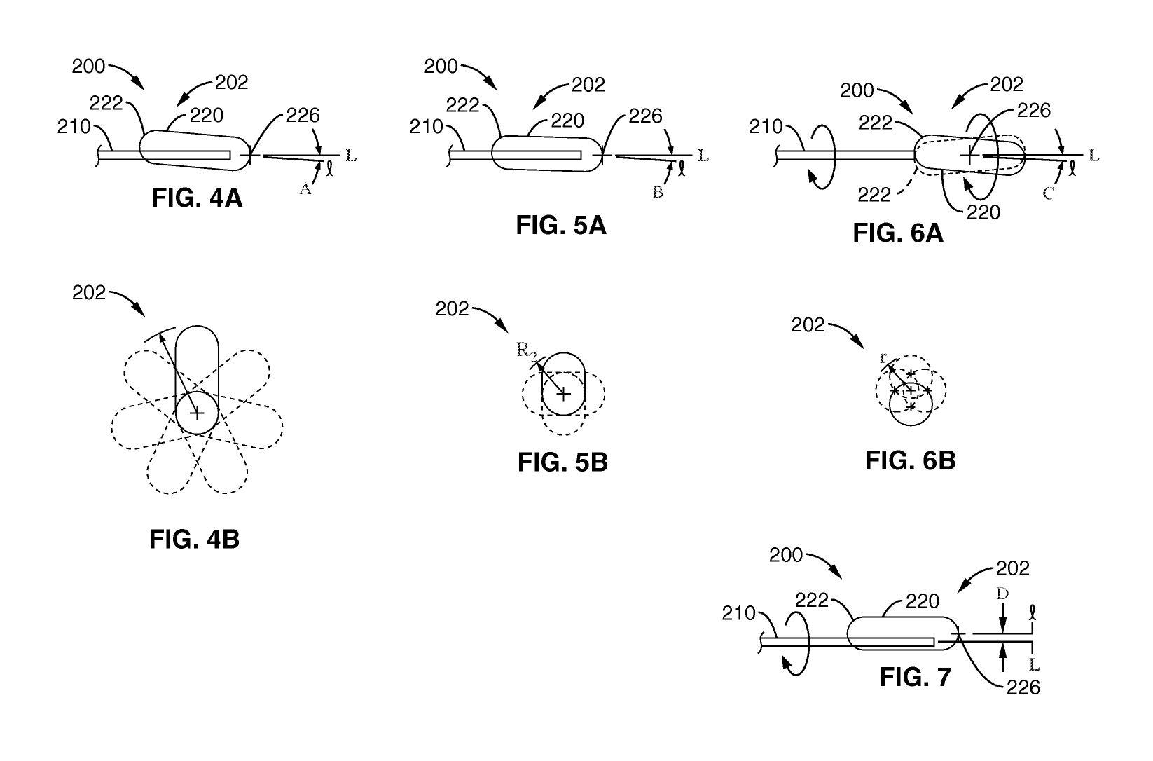

Various different tip configurations are contemplated. For example, as shown in FIG. 4A, wire 200 includes a core wire 210 that extends within a metal hypotube 220 and is canted by forcing the hypotube 220 to one side against the core wire 210 on the proximal end 222, and positioning the distal end 226 of the hypotube 220 to be substantially centered along longitudinal axis L of the core wire 210. This yields a canted enlarged member 202 as the distal tip of the wire 200 that exhibits a conical pattern of rotation, as shown in an illustrative end view in FIG. 4B about a radius R1 that is defined by the distance proximal end of hypotube 220 is offset from the central axis of rotation along longitudinal axis L.

However, this may be modified, such as along a more gentle, slight angle B as shown in FIGS. 5A and B rotating around a reduced radius R2 about longitudinal axis L. Moreover, enlarged member 202 may also be canted in such a manner that its distal end 226 is not positioned along longitudinal axis L and thus also rotates along a circumferential pattern about axis L. This is illustrated for example in FIGS. 6A and B. Moreover, rather than canting the angle of the enlarged member 202, it may instead entail a longitudinal axis I that is parallel to longitudinal axis L of rotation, but which longitudinal axis I is offset by a distance D from longitudinal axis L. This is illustrated for example in FIG. 7, wherein core wire 210 is forced against one wall between proximal and distal ends 222, 224 of the hypotube 220. In any event, the hypotube 220 may be for example similar to radiopaque markers, e.g. constructed from gold or platinum, and may be soldered, welded, adhesively bonded, or other wise secured at its proximal and distal ends 222, 224 to core wire 210.

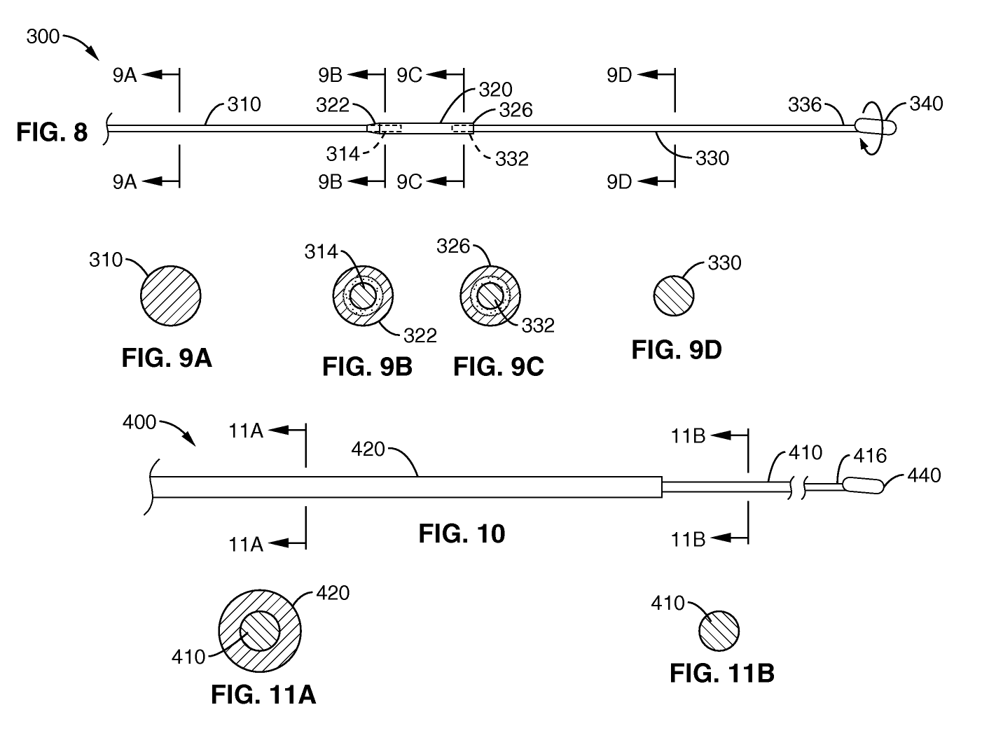

Core wire 210 may have many different constructions, two particular embodiments of which are shown for the purpose of illustration variously throughout FIGS. 8 to 11B.

More specifically, FIG. 8 shows a wire 300 constructed as follows. A stainless steel proximal core wire 310 is secured at a distal end portion 314 thereof into a proximal end 322 of a hypotube 320, and further including a distal core wire 330 of nickel-titanium superelastic alloy that has a proximal end 332 secured within the distal end 326 of the transition hypotube 320 and has a distal end 336 that is secured to the enlarged tip 340. In one beneficial mode the hypotube 320 is nickel-titanium alloy, and is secured to the proximal and distal core wires 310, 330 such as, for example by solder, welding, adhesive bonds, swaging, or other suitable known methods. Various cross sections of the portions of this wire 300 embodiment are variously shown in FIGS. 9A-D for the purpose of further illustration.

FIG. 10 shows a swaged wire 400 as another embodiment, having a stainless steel outer hypotube 420 swaged down over an internal core wire 410 constructed from a nickel-titanium superelastic alloy. The stainless steel hypotube 420 terminates so that only the nickel titanium alloy core wire 410 extends to the distal end portion 416 where the enlarged tip 440 is to be secured. This is further illustrated in FIGS. 11A-B.

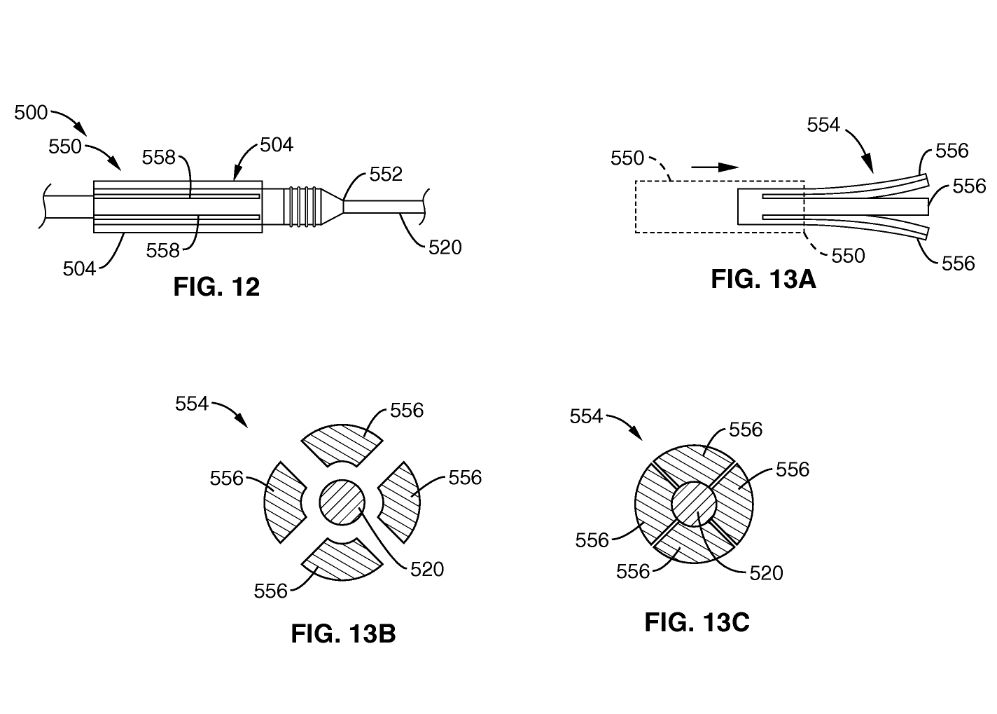

FIG. 12 shows one embodiment for adapting the wire 520 aspect of the sheath/wire assembly 500 for spinning rotation, and shows a proximal adapter 550 that is described as follows. Proximal wire adapter 550 includes a distal nose 552 that rotates with a threaded housing so as to advance or retract coaxially over a collet assembly 554 (see FIG. 13A) that includes a plurality of circumferentially oriented, radially biased longitudinal splines 556. This rotation and resulting longitudinal movement over the collet 554 actuates the collet 554 between radially open and closed conditions corresponding with relative radial locations of the splines 556, respectively, over the wire. Cross sections of the collet 554 in the open and closed conditions, and respective positions of the splines 556, are shown in FIGS. 13B-C, respectively. In any event, this is done with sufficient holding force to enable the proximal coupler 550 to be coupled into a rotating motor housing and to rotate the wire without excess and undesirable slippage at the coupler-wire interface.

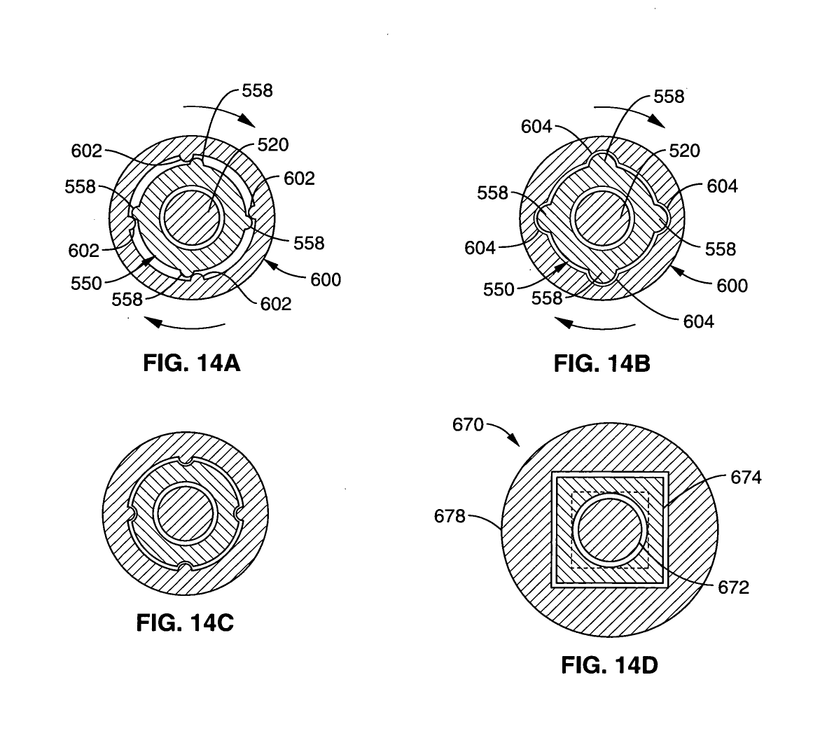

Proximal coupler 550 thereafter is inserted into a mating coaxial housing 600 in a motor actuator unit, as shown in cross section, for example, in FIG. 14A. The embodiment of FIG. 14A operates as follows. Outer housing 600 has ribs 602 that, during rotation of outer housing 600, mechanically abut exterior ribs 558 on collet adapter 550. By rotating the housing 600, the mechanical interference between the abutting ribs 558, 602 forces the collet coupler 550 to rotate with the outer housing 600. Many other embodiments are also contemplated and acceptable as apparent to one of ordinary skill. For example, various particular embodiments are shown in FIGS. 14B-D, wherein ribs 602 are variously replaced with recesses 604 such that a rib 558 on the opposing surface of collet coupler 550 is seated to allow for the interference during rotation. FIG. 14C shows an opposite relationship between components as another embodiment. Other keyed fittings are also contemplated, such as in the interfacing assembly 670 exemplified in FIG. 14D with a square keyhole type of interface between a proximal coupler 674 and outer housing 678. This type of interface may also apply to the interface of the proximal coupler 674 and internal wire 672, which may be “coined” to also have a square geometry (shown in shadow for illustration).

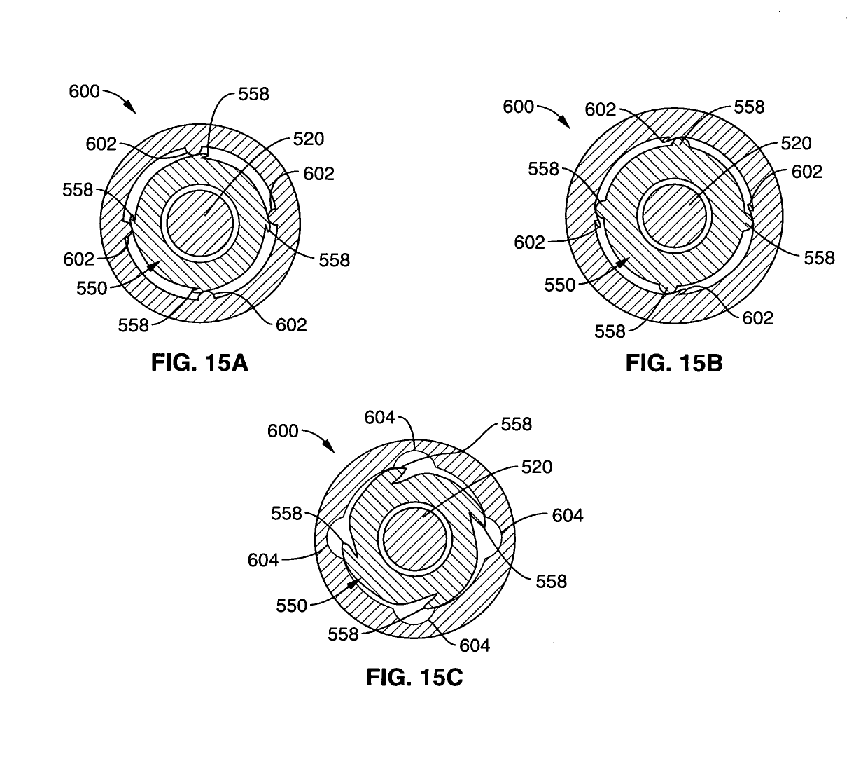

The proximal coupling may also be adapted to “give” or “slip” at desired amounts of torque, generally considered a safety feature to prevent overtorquing when the tip is stuck in a tight CTO and that might cause a failure such as stress kink or wire or bond breakage during adverse conditions of use. One mode for achieving such slippage provides the mechanical interface junctions with a controlled ability to “yield” and thus break the interference at a particular force level. This for example may be achieved by providing the ribs of known material with desired flexibility which at the dimension provided will yield predictably at the desired force. Various illustrative examples of proximal couplings to the motor housing where interfacing ribs are shown under yield during mechanical “slippage” at a particular force are provided at FIGS. 15A-C.

Various rotational actuator assemblies may be used according to the embodiments, as would be apparent to one of ordinary skill, and may be similar for example to other previously disclosed rotational actuators for other crossing guidewire attempts, or for various of the previously disclosed rotational atherectomy actuators. Therefore, the rotational actuator assemblies herein shown for the present embodiments are provided primarily in schematic form, and generally include a rotational housing coupled to a motor drive unit. However, in one beneficial embodiment not shown, the controlled rotational housing engaged with the wire proximal coupler is further coupled to a mechanical clutch mechanism associated with the motor of the actuator. The clutch mechanism may be mechanically constructed to slip at the predetermined rotational resistance force level. Or, electric circuitry may be adapted to automatically cut the motor or actuate the clutch at a predetermined force level, such as at particular current, voltage, or power levels associated with maintaining a particular speed. Further, a sensor may be included in the rotational actuator assembly and a control unit may be coupled to the motor and can be programmed to shut off the motor, or actuate a clutch, at a predetermined sensed force level.

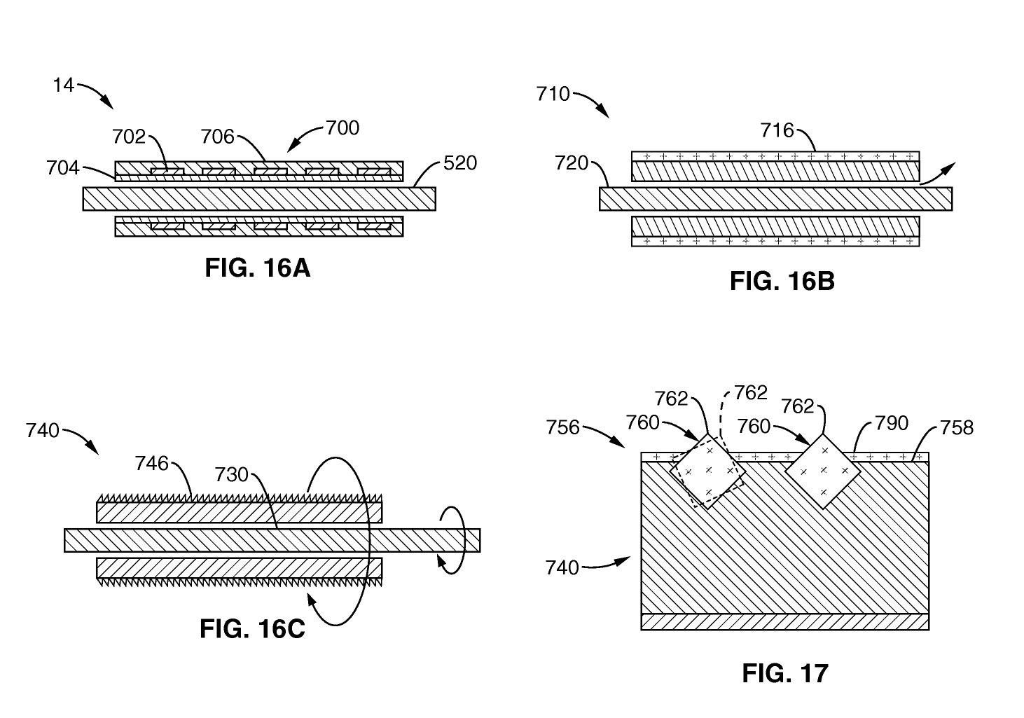

The outer tubular sheath feature of the various aspects, modes, and embodiments herein shown and described may have many different constructions and be suitable for use in the system as herein described. However, one particular beneficial embodiment is shown for example in FIG. 16A, and includes a composite wall 700 having a wire reinforcement 702 (e.g. wound flat ribbon) over an inner liner 704 and embedded within an outer jacket material 706. The liner beneficially is lubricious to the wire rotating within the lumen of the sheath, and may be for example a TEFLON® liner, high density polyethylene, graphite doped polymeric liner, or other suitable lubricious liner that will most typically be relatively thin, e.g. between about 0.001″ and about 0.005″, as would be appropriate to sufficiently provide the desired functional surface characteristic role in the composite. The outer jacket 706 material may be a heat shrinkable material that is shrunk down over the wire reinforcement 702 and inner liner 704, e.g. with an internal adhesive, or may be thermoplastic or thermoset and melted or dip coated onto the exterior surface. Examples include polyethylene, nylon, PEBAX®, polyurethane, polyimide, polyamide, polyolefin copolymer, or other suitable materials as known in the art. In further embodiments having distally reducing stiffness, the construction may change over the length of the sheath, such as by varying the materials to increasingly more flexible type distally, varying the pitch, dimension, or material of the reinforcing fiber, or by providing a tapered design. The reinforcement 702 may comprise a wound reinforcing ribbon, which may be for example a nickel titanium alloy in a superelastic state. In one beneficial embodiment, such superelastic ribbon is treated or “trained” to have its memory state in the wound configuration to enhance resistance to ovalization during bending or under the radial forces within a tight CTO lesion. Or, stainless steel ribbon may be used, which generally has a greater stiffness to resist crushing under forces in the lesion. Other suitable materials or constructions such as other metal ribbons, round wires, or fibers such as nylon or KEVLAR® fibers may be used for the sheath reinforcement, though highly pliable fibers such as nylon or KEVLAR® are not considered as beneficial for resistance to radial crushing or ovalization.

A further beneficial embodiment is shown in FIG. 16B, wherein sheath 710 includes an outer lubricious coating 716 that is adapted to aid in the advancement of the outer sheath 710 through a delivery sheath (not shown) to the lesion and/or into and through a tight CTO lesion in conjunction with or independently to advancement of the inner rotating wire 720. Suitable coatings may include hydrophilic coating such as a hydrogel, or Silicone coating preparation may be used. Other coating materials may be provided as would be apparent to one of ordinary skill, and may include for example bioactive coating such as thrombolytic coatings, heparin, hirudin, TPA, streptokinase, urokinase, or the like. These particular types of coatings may assist in the ability to cross a CTO lesion where remnants of an occlusive clot in the last true lumen may be dissolved to help open the way through the lesion. Moreover, such agents may be delivered through the crossing assembly, such as for example through the coaxial space between the outer sheath and the internal wire near the rotating distal wire tip (bolded arrow)

Other external treatments and constructions for the outer sheath feature of the present embodiments are also contemplated as further embodiments. For example, the outer surface 746 may be made appropriately abrasive as shown in FIG. 16C, which may help break up surrounding tissue during axial advancement through a tight CTO. Or, such abrasion may be used to ablate the tissue of the CTO that tightly surrounds the sheathed wire assembly 730, such as by spinning the respective outer sheath 740 within the CTO lesion either together with or independently of the internal wire 730, as will be developed below.

In any event, one particular example of an outer layer that includes an abrasive coating 756 is shown in exploded detail of a radial cross section in FIG. 17. This illustrative embodiment includes diamond particles 760 that are partially embedded within the outer surface 758 of the outer coating layer 756 of sheath 750, such that they are secured in place but have sharp tips 762 extending outwardly from the surface 758. This may be done for example by sputtering or otherwise exposing the outer surface 758 to a powder preparation of the diamond chips, such as when the outer layer 756 is wet from heat melting or solvent dipping onto the outer sheath 740. Upon curing, various of the diamond particles 760 are secured in various orientations, one of which is exemplified in the FIG. 17 embodiment. By providing the diamonds 760 embedded within a suitably soft outer layer polymer material, they are also able to yield under mechanical force of ablation, which effectively reduces the angle of their cutting edges and thus softens their ablative effect and is believed to provide a smoother resulting surface in the ablated result. For example, abrasive materials in flexible coatings have been previously disclosed for use in micropolishing other surfaces in industrial applications, such as for example internal bores of piston housings, with finer resulting surface finishes observed than is achievable with other techniques using abrasion on hard surfaces.

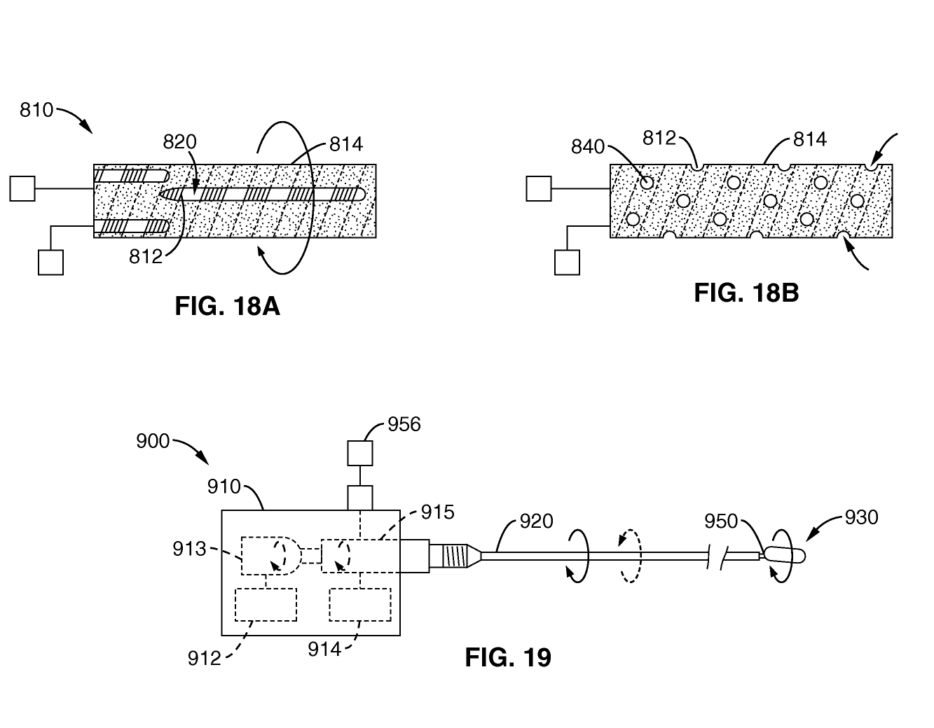

The various sheath constructions and coatings just described are exemplary and may be combined in various combinations or otherwise modified or replaced with other outer structures or materials. One such further embodiment uses an adhesive or other bonding layer material to bond an abrasive material onto the outer surface of the outer jacket layer of the sheath, rather than embedding the abrasive material into the outer layer wall material. Another beneficial combination of the previous embodiments described is illustrated by a sheath 740 that includes a lubricious outer coating 790 together with abrasive particles 760, as further shown in the FIG. 17 embodiment. For example, after the abrasive particles 760 are coated onto or partially embedded within the outer surface 758 of the outer sheath layer 756, the lubricious coating layer 790 may be applied. In one further variation, the lubricious coating does not bind to the diamond particles, but does coat onto the outer tube surface between the abrasive particles and may even bind there. This combination allows for the ability to ablate with the outer surface, as well as provide enhanced lubricity for the outer sheath to move across and through the CTO lesion material.

Where the outer sheath is used for rotational ablation of surrounding CTO lesion tissue, ports may be provided into the coaxial space between the outer sheath and internal wire, which space may be coupled to a vacuum source for suction removal of the ablated material. One example of such an arrangement is shown in FIG. 18A, where a groove-shaped port 820 is formed through the polymeric wall 814 of the outer sheath 810 but the reinforcing wire 812 is left in tact. This allows for a substantially continuous linear suction area along the grooved port 820 that is able to span a wide length of the adjacent blockage tissue during rotation and without substantial loss of tubular wall integrity due to the intact reinforcing member(s) 812. This may be performed for example by use of laser light of certain frequency that the polymer is ablated along the groove line but the metal reinforcement is not substantially affected. Alternatively, discrete ports 840 may be formed along the spacing between adjacent winds of the reinforcing ribbon, as shown in FIG. 18B. Or, the outer sheath may not include such reinforcing ribbon and port placement need not be so exact as to be located between windings.

As introduced above, the outer sheath feature of the various aspects, modes, and embodiments herein shown and described may be rotated with or independently of the respective inner wire feature that cooperates with the outer sheath in an overall functional system and method for crossing CTO's. One exemplary system 900 with an actuator assembly 910 for rotating the wire 920 and outer sheath 940 is shown schematically in FIG. 19. Here a proximal actuator assembly 910 includes first and second motors 912, 914 that rotate the wire 920 and outer sheath 940 separately via rotational couplers 913, 915, respectively. These motor driven rotational couplings within actuator assembly 910 may be rotated at same speeds and directions. Or, they may be rotated in opposite directions, as illustrated in shadow arrow in the figure. A suction port 956 may be coupled to the coaxial space 950 between the wire 920 and the outer sheath 940, as shown schematically to remove debris from the ablation. This port 956 and channel 950 may also be used for delivery of bioactive agent, as introduced above (or an additional fluid communication lumen may be provided so as to provide both suction and fluid delivery features).

By allowing the sheath to rotationally ablate the radially surrounding blockage tissue, resistance to additional advancement, e.g. through particularly long lesions, is reduced. In addition, a pilot hole is thus made through the lesion which may assist in the ability to later deliver another treatment device such as angioplasty balloon, stent assembly, or atherectomy assembly, into and through the CTO lesion. This is particularly useful for embodiments where the sheath may thereafter be removed with the inner wire left in place, and the treatment device is replaced over the wire through the pilot hole for treatment. This may also be particularly helpful in the case of relatively long CTO lesions in the peripheral vasculature, in particular in the legs (e.g. femoral arteries, SFA, etc.). For example, for outer sheaths of appropriately chosen outer diameters, the ablated pilot channel may be just about equal to or slightly greater than the profile of the treatment device to be positioned therein. Accordingly, it is further contemplated that a kit is provided that includes the outer sheath/wire assemblies herein described, together with a treatment device chosen for subsequent use in the pilot hole to be formed by the CTO assembly.

It is also contemplated that rotational ablation with the outer sheath may initiate with a large portion of the outer sheath located proximally of the lesion as the sheath/wire assembly is continued to advance through the lesion. Therefore, a second outer protective jacket may be provided over the first outer sheath and positioned just proximally against the lesion to protect proximal vessel wall from the proximal abrasive outer surfaces of the spinning assembly.

This disclosure variously describes the embodiments in terms of systems, assemblies, or devices for treatment of CTO lesions. While combinations of the components of such embodiments are highly beneficial, it is contemplated that each individual component alone may be highly beneficial, such as for example by virtue of their ability to be made and/or sold separately to be later interfaced with the other components. Moreover, to the extent various of the embodiments provide primarily the ability to place a guide rail across and through a CTO lesion and into a native downstream vessel lumen, such embodiments are nevertheless considered “treatment” systems or assemblies to the extent that they provide a mechanism by which recanalization or other treatment may be performed.

The invention has been discussed in terms of certain preferred embodiments. One of skill in the art will recognize that various modifications may be made without departing from the scope of the invention. Although discussed primarily in terms of crossing and treating CTO lesions, it should be understood that the embodiments could be used for other applications, such as other vascular blockages that do not qualify as CTO's, or other blockages in other body lumens or spaces. In addition, while particular cooperating or adjunctive treatment or other accessory devices are described for use in conjunction with the present embodiments, other modifications are contemplated as would be apparent to one of ordinary skill. Moreover, while certain features may be shown or discussed in relation to a particular embodiment, such individual features may be used on the various other embodiments of the invention.

1-2. (canceled)

3. A medical device system for conducting a medical procedure related to a medical condition within a body of a patient, comprising:
a first mechanically actuated device comprising a catheter with a first elongate body having a first proximal end portion and a first distal end portion; and
a second mechanically actuated device comprising a second elongate body with a second proximal end portion and a second distal end portion;
wherein a lumen extends within the first actuated device between a proximal port and a distal port that is located at the first distal end portion;
wherein the second actuated device is located at least in part within the lumen with the second distal end portion extending from the lumen through the distal port in a delivery configuration; and
wherein in the delivery configuration the first and second distal end portions are adapted to be delivered across a resistance to a location within the patient's body with the first and second proximal end portions, respectively, extending externally from the patient.

4-8. (canceled)

9. The system of claim 3, wherein:
the second mechanically actuated device comprises a guide wire;
wherein at least a section of the second distal end portion extends along a first longitudinal axis with a first outer diameter; and
wherein a distal tip section with a distal tip is located on the second distal end portion of the guide wire.

10-14. (canceled)

15. The system of claim 9, wherein:
the distal tip section of the guide wire comprises a second longitudinal axis between its proximal end and distal tip and that is angled or off-set relative to the first longitudinal axis;
the second elongate body of the guide wire is configured to be rotatably disposed at least in part within the lumen in a crossing configuration with the distal tip section of the guide wire extended externally of the lumen distally from the distal port;
the guide wire is sufficiently torquable such that upon rotation of the second proximal end portion externally of the patient's body sufficient torque is transmitted to the distal tip section at a CTO location within the patient's body so as to rotate the distal tip section about the first longitudinal axis of the guide wire's distal end portion; and
wherein the first elongate tubular body of the catheter and the guide wire are adapted to cooperate in coordinated advancement across the CTO in the crossing configuration.

16. The system of claim 15, wherein the first elongate tubular member is constructed so as to substantially inhibit resistance of the CTO on the torque transmission from the guide wire's second proximal end portion to the guide wire's distal tip section during the coordinated advancement of the guide wire and elongate tubular member through the CTO in the crossing configuration.

17-18. (canceled)

19. The system of claim 9, wherein each of the guide wire and catheter is coupled to a rotational actuator.

20. The system of claim 19, wherein each of the guide wire and catheter is rotationally actuated independently of the other.

21. (canceled)

22. The system of claim 15, wherein:
the first longitudinal axis crosses the second longitudinal axis about at the distal tip of the guide wire's distal tip section;
the proximal end of the distal tip section of the guide wire is offset from the first longitudinal axis; and
wherein upon rotation of the guide wire the radially off-set proximal end of the distal tip section rotates around the first longitudinal axis about a radius R, and the distal end remains substantially centered on the first longitudinal axis.

23. The system of claim 15, wherein:
the second longitudinal axis crosses the first longitudinal axis between the proximal end and the distal tip of the distal tip section.

24-25. (canceled)

26. The system of claim 3, wherein:
the first distal end portion of the catheter comprises a substantially tubular member with an ablative outer surface that is rotationally ablative to CTO tissue.

27. The system of claim 26, wherein the ablative outer surface does not comprise a substantially radially enlarged member.

28-31. (canceled)

32. The system of claim 9, wherein the distal end portion of the guide wire comprises a nickel titanium core wire.

33. The system of claim 32, wherein the second proximal end portion of the guide wire comprises a stainless steel alloy material.

34. The system of claim 33, wherein the second proximal end portion of the guide wire comprises a stainless steel hypotube coupled to the nickel titanium second distal end portion.

35. (canceled)

36. The system of claim 3, further comprising:
a delivery sheath with a delivery lumen and that is adapted to be delivered to a CTO location within a patient's body;
wherein the catheter is adapted to be delivered to the CTO location through the delivery lumen.

37. (canceled)

38. The system of claim 3, wherein the catheter is adapted to ablate a pilot lumen through a CTO lesion of at least about 10 centimeters and to recanalize only about ⅓ or less of the cross-sectional area of the CTO lesion.

39. (canceled)

40. The system of claim 9, further comprising a second interventional catheter that is adapted to track over the guide wire and substantially recanalize the CTO lesion crossed with the guide wire and catheter of the system.

41. The system of claim 9, wherein the catheter is removable from the guide wire after crossing the CTO lesion.

42. The system of claim 9, wherein the catheter and guide wire are each tapered with a distally reducing outer diameter.

43-44. (canceled)

45. The medical device system of claim 9, further comprising:
a catheter actuator;
wherein the catheter is configured to be actuated by the catheter actuator; and
a wire actuator;
wherein the guide wire is configured to be actuated by the wire actuator;
wherein the second distal end portion of the guide wire has a section with a first outer diameter, and the distal tip section of the guide wire has a second outer diameter that is radially enlarged relative to the first outer diameter;
wherein the catheter is adapted to moveably engage the guide wire in a crossing configuration with the guide wire extending within the lumen and through the proximal and distal ports with the enlarged distal tip section located externally of the lumen distally beyond the distal port; and
wherein the actuated catheter and the actuated guide wire are configured to advance across a CTO substantially together in the crossing configuration.

46. A method for conducting a medical procedure related to a medical condition within a body of a patient, comprising:
mechanically actuating a first device comprising a catheter with a first elongate body having a first proximal end portion and a first distal end portion;
mechanically actuating a second device comprising a second elongate body with a second proximal end portion and a second distal end portion;
positioning the second mechanically actuated device at least in part within a lumen of the first mechanically actuated device that extends within the first mechanically actuated device between a proximal port and a distal port at the first distal end portion;
extending the second distal end portion of the second mechanically actuated device from the lumen through the distal port in a delivery configuration; and
delivering the first and second distal end portions in the delivery configuration across a resistance to a location within the patient's body with the first and second proximal end portions, respectively, extending externally from the patient.
